SEQUENCE LISTING (6 sequences)

SEQ ID NO: 1 - protein, 9 aa, synthetic construct
  MOD_RES 2: note = D amino acid
  MOD_RES 5: note = D amino acid
  MOD_RES 8: note = D amino acid
  VARIANT 1: note = can be substituted with any L amino acid that is not proline
  VARIANT 4: note = can be substituted with any L amino acid that is not proline
  VARIANT 7: note = can be substituted with any L amino acid that is not proline
EHPEHPEHP

SEQ ID NO: 2 - protein, 9 aa, synthetic construct
  MOD_RES 2..3: note = D amino acid
  MOD_RES 5..6: note = D amino acid
  MOD_RES 8..9: note = D amino acid
  VARIANT 3: note = can be any alpha D amino acid that is not proline
  VARIANT 6: note = can be any alpha D amino acid that is not proline
  VARIANT 9: note = can be any alpha D amino acid that is not proline
DHMDHMDHM

SEQ ID NO: 3 - protein, 6 aa, synthetic construct
  VARIANT 1: note = (3-(4-Pyridyl)-alanine
  VARIANT 2: note = beta-Homoproline
  VARIANT 3: note = D-alpha-Aminobutyric acid or any D amino acid that is not proline
  VARIANT 6: note = D-alpha-Aminobutyric acid or any D amino acid that is not proline
  VARIANT 5: note = beta-Homoproline
  VARIANT 4: note = (3-(4-Pyridyl)-alanine
APXAPX

SEQ ID NO: 4 - skipped

SEQ ID NO: 5 - protein, 12 aa, synthetic construct
  MOD_RES 1: note = D amino acid
  MOD_RES 3..4: note = D amino acid
  MOD_RES 6: note = D amino acid
  MOD_RES 8: note = D amino acid
  MOD_RES 11: note = D amino acid
  VARIANT 1: note = can be substituted to any D amino acid that is not proline
  VARIANT 3..4: note = can be substituted to any D amino acid that is not proline
  VARIANT 6: note = can be substituted to any D amino acid that is not proline
  VARIANT 8: note = can be substituted to any D amino acid that is not proline
  VARIANT 2: note = can be substitutes to any L amino acid that is not proline
  VARIANT 7: note = can be substitutes to any L amino acid that is not proline
  VARIANT 9..10: note = can be substitutes to any L amino acid that is not proline
  VARIANT 12: note = can be substitutes to any L amino acid that is not proline
ANKHPEANKHPE

SEQ ID NO: 6 - protein, 10 aa, synthetic construct
  MOD_RES 1..2: note = D amino acid
  MOD_RES 4: note = D amino acid
  MOD_RES 8: note = D amino acid
  MOD_RES 10: note = D amino acid
  VARIANT 4: note = can be substituted to any D amino acid that is not proline
  VARIANT 9: note = can be substituted to any L amino acid that is not proline
PPKVEPPKVE

(19) United States
(12) Patent Application Publication
(10) Pub. No.: US 2023/0374075 A1
(43) Pub. Date: Nov. 23, 2023

(54) Structured Symmetric Cyclic Peptides as Ligands for Metal Organic Frameworks

(71) Applicant: University of Washington, Seattle, WA (US)

(72) Inventors: David Baker, Seattle, WA (US); Ryanne Ballard, Seattle, WA (US); Asim Bera, Seattle, WA (US); Christine Kang, Seattle, WA (US); Alex Kang, Seattle, WA (US); Xinting Li, Seattle, WA (US); Hannah Nguyen, Seattle, WA (US); Meerit Said, Seattle, WA (US); Patrick Salveson, Seattle, WA (US); William H. Sheffler, Seattle, WA (US); Shunzhi Wang, Seattle, WA (US)

(21) Appl. No.: 18/319,343

(22) Filed: May 17, 2023

Related U.S. Application Data
(60) Provisional application No. 63/343,773, filed on May 19, 2022.

(51) Int. Cl. C07K 7/64; C07K 17/14; B01J 31/16

(52) U.S. Cl. CPC C07K 7/64; C07K 17/14; B01J 31/1691

(57) ABSTRACT

Cyclic peptides including the amino acid sequence selected from SEQ ID NO:1-6, multimers thereof, and metal organic frameworks including the cyclic peptides or multimers thereof are provided.

### CROSS REFERENCE

This application claims priority to U.S. Provisional Patent Application Ser. No. 63/343,773 filed May 19, 2022, incorporated by reference herein in its entirety.

### STATEMENT OF GOVERNMENT RIGHTS

This invention was made with government support under Grant No. HDTRA1-19-1-0003, awarded by the Defense Threat Reduction Agency. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on May 11, 2023 having the file name “22-0804-US.xml” and is 20,042 bytes in size.

### BACKGROUND

Despite remarkable advances in the assembly of highly structured coordination polymers and metal-organic frameworks (MOFs), the rational design of such materials using more conformationally flexible organic ligands such as peptides remains challenging.

### SUMMARY

In one aspect, the disclosure provides cyclic peptides, comprising a sequence selected from the group consisting of:
(a) EhPEhPEhP (SEQ ID NO:1), wherein E can be substituted with any L amino acid that is not proline,
(b) DhmDhmDhm (SEQ ID NO:2), wherein m can be any alpha D amino acid that is not proline,
(c) (3-(4-Pyridyl)-alanine-β-Homoproline-D-α-Aminobutyric acid-3-(4-Pyridyl)alanine-β-Homoproline-D-α-Aminobutyric acid), wherein D-α-Aminobutyric acid can be substituted with any D amino acid that is not proline (SEQ ID NO:3),
(d) (3-(4-Pyridyl)-alanine-1,2,3,4-tetrahydroisoquinoline-3-carboxylic acid-3-Aminobutanoic acid-3-(4-Pyridyl)-alanine-1,2,3,4-tetrahydroisoquinoline-3-carboxylic acid-3-Aminobutanoic acid wherein 3-aminobutanoic acid can be substituted to any R amino acid such as β-phenylalanine, β-alanine, or 3-Aminoisobuteric acid);
(e) aNkhPeAnKHpE (SEQ ID NO:5), or salt thereof, wherein a, k, h, e, and n can be substituted to any D amino acid that is not proline; and wherein N, A, K, H, and E can be substitutes to any L amino acid that is not proline; and (ii) a metal ion; and
(f) ppKvEPPkVe (SEQ ID NO:6), or salt thereof, wherein v can be substituted to any D amino acid that is not proline, and V can be substituted to any L amino acid that is not proline;
wherein single letter amino acid residues in upper case are L amino acids, and single letter amino acid residues in lower case are D amino acids.

In another aspect, the disclosure provides multimers comprising 2 or more copies of an identical cyclic peptide of the disclosure. In some embodiments, the multimer further comprises a metal ion.

In a further aspect, the disclosure provides metal-organic frameworks (MOF), comprising:
(a) a multimer comprising (i) at least two copies of cyclic peptide EhPEhPEhP (SEQ ID NO:1) or salt thereof, wherein E can be substituted with any L amino acid that is not proline, and (ii) a metal ion;
(b) a multimer comprising (i) at least two copies of cyclic peptide DhmDhmDhm (SEQ ID NO:2), or salt thereof, wherein m can be any alpha D amino acid that is not proline, and (ii) a metal ion;
(c) a multimer comprising (i) at least two copies of cyclic peptide (3-(4-Pyridyl)-alanine-β-Homoproline-α-Aminobutyric acid-3-(4-Pyridyl)-alanine-β-Homoproline-α-Aminobutyric acid), or salt thereof, wherein D-α-Aminobutyric acid can be substituted with any D amino acid that is not proline (SEQ ID NO:3), and (ii) a metal ion;
(d) a multimer comprising (i) at least two copies of cyclic peptide (3-(4-Pyridyl)-alanine-1,2,3,4-tetrahydroisoquinoline-3-carboxylic acid-3-Aminobutanoic acid-3-(4-Pyridyl)-alanine-1,2,3,4-tetrahydroisoquinoline-3-carboxylic acid-3-Aminobutanoic acid), or salt thereof; wherein 3-aminobutanoic acid can be substituted to any R amino acid such as β-phenylalanine, β-alanine, or 3-Aminoisobuteric acid (SEQ ID NO:4), and (ii) a metal ion;
(e) a multimer comprising (i) at least two copies of cyclic peptide aNkhPeAnKHpE (SEQ ID NO:5), or salt thereof, wherein a, k, h, e, and n can be substituted to any D amino acid that is not proline; and wherein N, A, K H, and E can be substitutes to any L amino acid that is not proline; and (ii) a metal ion; or
(f) a multimer comprising (i) at least two copies of cyclic peptide ppKvEPPkVe (SEQ ID NO:6), or salt thereof, wherein v can be substituted to any D amino acid that is not proline. V can be substituted to any L amino acid that is not proline; and (ii) a metal ion;
wherein single letter amino acid residues in upper case are L amino acids, and single letter amino acid residues in lower case are D amino acids.

### FIGURE LEGENDS

FIG. 1. Computational method for designing metal mediated 3D lattices from rigid symmetric peptide building blocks. (a) Rotamers of metal coordinating residues such as histidines are sampled on symmetric peptide backbones. (b) Symmetric metal mediated interactions between pairs of peptides are sampled according to standard coordination geometry. (c) Peptide arrangements with dihedral angles between the axis of symmetry of the peptide and the axis of symmetry of the metal compatible with ideal lattice geometry for particular space groups are selected. (d) Examples of modeled lattices in space groups P4₃32, P4₁32, P23, and I2₁3. (e) Close up view of the metal coordination for each lattice.

FIG. 2. Structure of the C3-1-Co²⁺ crystal. (a) Metal coordination in the design model. (b) The designed P4₃32 lattice. (c) Crystal structure of the C3-1 ligand aligned with the design model. (d) A single layer of Co²⁺-C3-1 2D sheet with C3 symmetry. (e) Three histidine and three waters coordination of C3-1 in the crystal structure. (f) View along the b axis showing six layers of 2D planes stacked in a twisted way (dashed curves).

FIG. 3. Structure of the C3-2: Zn²⁺ crystal. (a) Design model (left) and crystal structure (right). (b) Adjacent Zn-C3-2 1D chains interact via dispersion interactions. (c) Metal coordinating peptide chain (d) Views of the Zn-C3-1 crystal along the b axis.

FIG. 4. Structures of lattices formed by C2 pyridine containing peptides. (a) Computational model of C2-1 ligand. (b) Crystal structure of the C2-1 ligand which is 4.7 kCal/mol higher in energy than the design model. (c) A zoomed view of the Zn²⁺-C2-1 1D chain (two coordinated water molecules are omitted). (d) View into 1D chain along the b axis. (e) Computational model of C2-2 ligand. (f) Crystal structure of the C2-2 ligand which is 3.4 kCal/mol higher in energy than the design model. (g) A zoomed view of the Zn²⁺-C2-1 1D chain (two coordinated water molecules are omitted). (h) View into 1D chain along the c axis of the crystal in P12₁1 space group. (i) View along the a axis of the crystal in C121 space group.

FIG. 5. Lattices formed by cyclic peptides with S2 symmetry. (a) S2-1 ligand. (b) A single Cu²⁺ ion is coordinated with two peptides via two lysines and two glutamates in a square planar geometry. (c) View along the b axis highlighting a linearly coordinated S2-1 chain (dashed rectangle). (d) View along the c axis. (e) S2-2 ligand. (f) 1D open channel has an inner diameter of ˜1 nm. (g) Zn²⁺ ions are tetrahedrally coordinated with solvents. (h) View along the c axis showing hexagonal shaped open channels occupied by Zn²⁺ ions.

FIG. 6. Predicted energy landscape for each designed peptide. The first column shows the designed conformation, while the second column shows the energy landscape calculated using either AIMNet (a-b) or Rosetta™ (c-f). Backbone RMSD is calculated to the designed conformation on the left. (a) AIMNet energy landscape for C2-1. (b) AIMNet energy landscape for C2-2. (c) Rosetta™ energy landscape for C3-2. (d) Rosetta™ energy landscape for S2-1. (e) Rosetta™ energy landscape for S2-2. (f) Rosetta™ energy landscape for C3-1.

FIG. 7. Alignment of C2-2 apo design to crystal structure with 0.5 Å Cα RMSD.

FIG. 8. Mercury calculated void volume shown for each peptide crystal. (a) C2-1 crystal shown along the c axis. (b) C2-2 crystal 1 (left) and crystal 2 (right) shown along the b axis. (c) C3-1 crystal shown along the b axis. (d) C3-2 crystal shown along the b axis. (e) S2-1 crystal shown along the “a” axis. (f) S2-2 crystal shown along the c axis.

FIG. 9. View along the b axis of C2-2 crystal. (a) Zoomed in view of the solvent accessible cavity with partially coordinated zinc ions. (b) View of one pore going through the crystal lattice. (c) Multiple space units of C2-2 in the P12₁1 space group.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

All references cited are herein incorporated by reference in their entirety. As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Amino acid residues shown in upper case are L amino acids, and residues in lower case are D amino acids

All embodiments of any aspect of the disclosure can be used in combination, unless the context clearly dictates otherwise.

In one aspect, the disclosure provides cyclic peptides, comprising a sequence selected from the group consisting of:
(a) EhPEhPEhP (SEQ ID NO:1), wherein E can be substituted with any L amino acid that is not proline,
(b) DhmDhmDhm (SEQ ID NO:2), wherein m can be any alpha D amino acid that is not proline, where an alpha D amino acid is a D-amino acid with N-Calpha-C backbone as opposed to more than two carbons in the backbone,
(c) (3-(4-Pyridyl)-alanine-β-Homoproline-D-α-Aminobutyric acid-3-(4-Pyridyl)alanine-β-Homoproline-D-α-Aminobutyric acid) (SEQ ID NO:3), wherein D-α-Aminobutyric acid can be substituted with any D amino acid that is not proline,
(d) (3-(4-Pyridyl)-alanine-1,2,3,4-tetrahydroisoquinoline-3-carboxylic acid-3-Aminobutanoic acid-3-(4-Pyridyl)-alanine-1,2,3,4-tetrahydroisoquinoline-3-carboxylic acid-3-Aminobutanoic acid wherein 3-aminobutanoic acid can be substituted to any R amino acid such as β-phenylalanine, β-alanine, or 3-Aminoisobuteric acid);
(e) aNkhPeAnKHpE (SEQ ID NO:5), or salt thereof, wherein a, k, h, e, and n can be substituted to any D amino acid that is not proline; and wherein N, A, K, H, and E can be substitutes to any L amino acid that is not proline; and (ii) a metal ion; and
(f) ppKvEPPkVe (SEQ ID NO:6), or salt thereof, wherein v can be substituted to any D amino acid that is not proline, and V can be substituted to any L amino acid that is not proline;
wherein single letter amino acid residues in upper case are L amino acids, and single letter amino acid residues in lower case are D amino acids.

As described in the examples, the cyclic peptides can be used in generating the multimers and metal-organic frameworks (MOFs) as disclosed herein. MOFs comprising large peptide ligands with internal symmetry have not been previously explored, and the present disclosure provides the first structures of symmetric cyclic 6 to 12 residue peptide MOFs with both proper and improper symmetries (C2, C3, and S2), employing for metal-chelation histidine, cysteine, aspartate, glutamate, and noncanonical amino acids containing pyridine and DOPA side chains. Exemplary crystal structures of six peptide MOFs with different metals (Zn²⁺, Co²⁺, and Cu²⁺) and space groups (P1, P65, C121, P12₁1, R3, P4₁2₁2, and P-1) contain a rich variety of 1D and 2D metal-mediated structures with pore shapes and sizes ranging from 7% to 40% void volume. The large surface area and pore sizes of these peptide-metal lattices make them particularly useful, for example, in catalysis and sensing, and the wide variety of both natural and unnatural sidechains available allows facile customization of chemistry lining the pores and other structural features of the crystals.

In one embodiment, the cyclic peptide comprises EhPEhPEhP (SEQ ID NO:1), wherein E can be substituted with any L amino acid that is not proline. In various embodiments, 1, 2, or all 3 E residues may be substituted with any L amino acid that is not proline. In embodiments where 2 or 3 E residues are substituted, they may be substituted with the same L amino acid, or different L amino acids. In another embodiment the cyclic peptide is EhPEhPEhP (SEQ ID NO:1) and no E residues are substituted.

In another embodiment, the cyclic peptide comprises DhmDhmDhm (SEQ ID NO:2), wherein m can be any alpha D amino acid that is not proline. In various embodiments, 1, 2, or all 3 D residues may be substituted with any D amino acid that is not proline. In embodiments where 2 or 3 D residues are substituted, they may be substituted with the same D amino acid, or different D amino acids. In another embodiment the cyclic peptide is DhmDhmDhm (SEQ ID NO:2) and no D residues are substituted.

In a further embodiment, the cyclic peptide comprises (3-(4-Pyridyl)-alanine-β-Homoproline-D-α-Aminobutyric acid-3-(4-Pyridyl)-alanine-β-Homoproline-D-α-Aminobutyric acid) (SEQ ID NO:3), wherein D-α-Aminobutyric acid can be substituted with any D amino acid that is not proline. In one embodiment, one or both D-α-Aminobutyric acid can be substituted with any D amino acid that is not proline. In embodiments where both D-α-Aminobutyric acid residues are substituted, they may be substituted with the same D amino acid amino acid, or different D amino acid amino acids. In another embodiment, neither D-α-Aminobutyric acid is substituted.

In one embodiment, the cyclic peptide comprises 3-(4-Pyridyl)-alanine-1,2,3,4-tetrahydroisoquinoline-3-carboxylic acid-3-Aminobutanoic acid-3-(4-Pyridyl)-alanine-1,2,3,4-tetrahydroisoquinoline-3-carboxylic acid-3-Aminobutanoic acid, wherein 3-aminobutanoic acid can be substituted to any R amino acid such as β-phenylalanine, β-alanine, or 3-Aminoisobuteric acid). In one embodiment, one or both 3-aminobutanoic acid can be substituted. In embodiments where both 3-aminobutanoic acid residues are substituted, they may be substituted with the same R amino acid amino acid, or different β amino acid amino acids. In another embodiment, neither 3-aminobutanoic acid residue is substituted.

In another embodiment, the cyclic peptide comprises aNkhPeAnKHpE (SEQ ID NO:5), or salt thereof, wherein a, k, h, e, and n can be substituted to any D amino acid that is not proline; and wherein N, A, K, H, and E can be substituted to any L amino acid that is not proline. In various embodiments, 1, 2, 3, 4, or all 5 of residues a, k, h, e, and n may be substituted with any D amino acid that is not proline. In embodiments where 2, 3, 4, or 5 residues are substituted, they may be substituted with the same D amino acid, or different D amino acids. In no residues are substituted.

In a further embodiment, the cyclic peptide comprises ppKvEPPkVe (SEQ ID NO:6), or salt thereof, wherein v can be substituted to any D amino acid that is not proline.

The cyclic peptides may comprise additional linked moieties. In one embodiment, one or more substitutable residue on the cyclic peptide comprises an additional moiety attached via a side chain of the substitutable residue or a residue substituting for the substitutable residue. The substitutable residues are described above. In this embodiment, any suitable residue may substitute for the substitutable residue as appropriate for an intended purpose. In another embodiment, one or more substitutable residue is substituted with a lysine residue. In one such embodiment, one or more substitutable residue is substituted with a lysine residue and one or more lysine residue is conjugated to an additional moiety. The additional moiety may be any as suitable for an intended purpose. In various embodiments, the additional moiety may include but not limited to amino acids, nucleotides, polyethylene glycol (PEG), or fluorescent molecules, which can be used, for example, for solubilization, catalysis or sensing of different ligands of interest such as nerve agents.

In another embodiment, the disclosure provides multimers comprising 2 or more copies of an identical cyclic peptide of any embodiment or combination of embodiments disclosed above. In one embodiment, the multimers comprise a metal ion. As described in the examples, the inventors designed metal mediated 3D frameworks using peptide macrocycles of the disclosure with metal coordinating sidechains. Any metal ion may be used as suitable for an intended use. In certain embodiments, the metal ion is selected from the group consisting of Co²⁺. Zn²⁺, Fe³⁺, and Cu²⁺.

In a further embodiment, the disclosure provides metal-organic frameworks (MOF), comprising:
(a) a multimer comprising (i) at least two copies of cyclic peptide EhPEhPEhP (SEQ ID NO:1) or salt thereof, wherein E can be substituted with any L amino acid that is not proline, and (ii) a metal ion;
(b) a multimer comprising (i) at least two copies of cyclic peptide DhmDhmDhm (SEQ ID NO:2) or salt thereof, wherein m can be any alpha D amino acid that is not proline, and (ii) a metal ion;
(c) a multimer comprising (i) at least two copies of cyclic peptide (3-(4-Pyridyl)-alanine-β-Homoproline-α-Aminobutyric acid-3-(4-Pyridyl)-alanine-β-Homoproline-α-Aminobutyric acid) (SEQ ID NO:3), or salt thereof, wherein D-α-Aminobutyric acid can be substituted with any D amino acid that is not proline, and (ii) a metal ion;
(d) a multimer comprising (i) at least two copies of cyclic peptide (3-(4-Pyridyl)-alanine-1,2,3,4-tetrahydroisoquinoline-3-carboxylic acid-3-Aminobutanoic acid-3-(4-Pyridyl)alanine-1,2,3,4-tetrahydroisoquinoline-3-carboxylic acid-3-Aminobutanoic acid), or salt thereof; wherein 3-aminobutanoic acid can be substituted to any R amino acid such as β-phenylalanine, β-alanine, or 3-Aminoisobuteric acid, and (ii) a metal ion;
(e) a multimer comprising (i) at least two copies of cyclic peptide aNkhPeAnKHpE (SEQ ID NO:5), or salt thereof, wherein a, k, h, e, and n can be substituted to any D amino acid that is not proline; and wherein N, A, K H, and E can be substitutes to any L amino acid that is not proline; and (ii) a metal ion; or
(f) a multimer comprising (i) at least two copies of cyclic peptide ppKvEPPkVe (SEQ ID NO:6), or salt thereof, wherein v can be substituted to any D amino acid that is not proline, and V can be substituted to any L amino acid that is not proline; and (ii) a metal ion;
wherein single letter amino acid residues in upper case are L amino acids, and single letter amino acid residues in lower case are D amino acids.

The metal-organic frameworks (MOFs) of the disclosure can be used, for example, in metal capture, hydrolysis of phospho-ester compounds including nerve agents (such as VX compound), and capture of gasses such as CO2. All embodiments of the cyclic peptides as disclosed herein are equally applicable to the MOFs of the disclosure.

In one embodiment of the MOFs of the disclosure, one or more substitutable residue on the cyclic peptide comprises an additional moiety attached via a side chain of the substitutable residue. As discussed above, the cyclic peptides may comprise additional linked moieties. In one embodiment, one or more substitutable residue on the cyclic peptide comprises an additional moiety attached via a side chain of the substitutable residue or a residue substituting for the substitutable residue. The substitutable residues are described above. In this embodiment, any suitable residue may substitute for the substitutable residue as appropriate for an intended purpose. In another embodiment, one or more substitutable residue is substituted with a lysine residue. In one such embodiment, one or more substitutable residue is substituted with a lysine residue and one or more lysine residue is conjugated to an additional moiety. The additional moiety may be any as suitable for an intended purpose. In various embodiments, the additional moiety may include but not limited to amino acids, nucleotides, polyethylene glycol (PEG), or fluorescent molecules, which can be used, for example, for solubilization, catalysis or sensing of different ligands of interest such as nerve agents.

Any metal ion may be used as suitable for an intended use of the MOFs. In certain embodiments, the metal ion is selected from the group consisting of Co²⁺. Zn²⁺, Fe³⁺, and Cu²⁺.

In one embodiment, the MOF comprises a multimer comprising (i) at least two copies of cyclic peptide EhPEhPEhP (SEQ ID NO:1) or salt thereof, wherein E can be substituted with any L amino acid that is not proline, and (ii) Co²⁺. In one such embodiment, the MOF comprises a crystal structure, optionally wherein in the crystal structure each Co²⁺ cation is octahedrally coordinated to three water molecules and three histidines from different peptides in a planar fashion, and the glutamates do not participate in coordination but fill the crystal pores.

In another embodiment, the MOF comprises a multimer comprising (i) at least two copies of cyclic peptide DhmDhmDhm (SEQ ID NO:2) or salt thereof, wherein m can be any alpha D amino acid that is not proline, and (ii) Zn²⁺. In one such embodiment, the MOF comprises a crystal structure, optionally wherein in the crystal structure each Zn²⁺ cation is internally coordinated with three histidines from one peptide and an aspartic acid from an adjacent peptide.

In a further embodiment, the MOF comprises a multimer comprising (i) at least two copies of cyclic peptide (3-(4-Pyridyl)-alanine-β-Homoproline-α-Aminobutyric acid-3-(4-Pyridyl)-alanine-β-Homoproline-α-Aminobutyric acid) (SEQ ID NO:3), or salt thereof, wherein D-α-Aminobutyric acid can be substituted with any D amino acid that is not proline, and (ii) Zn²⁺. In one such embodiment, the MOF comprises a crystal structure, optionally wherein in the crystal structure each Zn²⁺ cation is linked to two peptides through pyridine coordination while two water molecules fill the other positions for full tetrahedral coordination.

In one embodiment the MOF comprises a multimer comprising (i) at least two copies of cyclic peptide 3-(4-Pyridyl)-alanine-1,2,3,4-tetrahydroisoquinoline-3-carboxylic acid-3-Aminobutanoic acid-3-(4-Pyridyl)-alanine-1,2,3,4-tetrahydroisoquinoline-3-carboxylic acid-3-Aminobutanoic acid), or salt thereof; wherein 3-aminobutanoic acid can be substituted to any amino acid such as β-phenylalanine, β-alanine, or 3-Aminoisobuteric acid, and (ii) Zn²⁺. In one such embodiment, the MOF comprises a crystal structure, optionally wherein in the crystal structure each Zn²⁺ cation is tetrahedrally coordinated with two pyridine ligands and two water molecules.

In another embodiment, the MOF comprises a multimer comprising (i) at least two copies of cyclic peptide aNkhPeAnKHpE (SEQ ID NO:5), or salt thereof; wherein a, k, h, e, and n can be substituted to any D amino acid that is not proline; and wherein N, A, K H, and E can be substitutes to any L amino acid that is not proline; and (ii) Zn²⁺. In one such embodiment, the MOF comprises a crystal structure, optionally wherein in the crystal structure each Zn²⁺ cation occupies empty open channels in the crystal and are not coordinated to any of the metal-binding residues.

In a further embodiment, the MOF comprises a multimer comprising (i) at least two copies of cyclic peptide ppKvEPPkVe (SEQ ID NO:6), or salt thereof, wherein v can be substituted to any D amino acid that is not proline. V can be substituted to any L amino acid that is not proline; and (ii) Cu²⁺. In one such embodiment, the MOF comprises a crystal structure, optionally wherein in the crystal structure each Cu²⁺ cation is coordinated between two peptides via two lysines and two glutamates in a square planar geometry.

The disclosure also provides methods for use of the MOF of any embodiment or combination of embodiments herein, including but not limited to binding of metal binding compounds.

The disclosure also provides method for designing MOFs and cyclic peptides that can form MOFs, comprising any steps or combinations of steps as disclosed in the examples.

### Examples

In an effort to make the design of highly structured coordination polymers and metal-organic frameworks (MOFs) fully programmable, we first developed a computational design method for generating metal mediated 3D frameworks using rigid and symmetric peptide macrocycles with metal coordinating sidechains. We solved the structures of six crystalline networks involving conformationally constrained 6 to 12 residue cyclic peptides with C2, C3 and S2 internal symmetry and three different types of metals (Zn²⁺, Co²⁺, or Cu²⁺) by single-crystal X-ray diffraction (XRD), which reveal how the peptide sequences, backbone symmetries, and metal coordination preferences drive the assembly of the resulting structures. In contrast to smaller ligands, these peptides associate through peptide-peptide interactions without full coordination of the metals, contrary to one of the assumptions underlying our computational design method. The cyclic peptides are the largest peptidic ligands reported to form crystalline coordination polymers with transition metals to date. The combination of high chemical diversity with synthetic accessibility makes them attractive for use in applications such as sensing, asymmetric catalysis, and chiral separation.

### Introduction

Metal-peptide frameworks reported to date involve short linear peptides (e.g. di- and tripeptides) Use of longer peptides as organic linkers in this way has been challenging because of their greater conformational flexibility.

Here we set out to explore the design of MOFs using these symmetric cyclic peptides with well-defined backbone structures as metal ligands. These compounds have potential advantages over previous peptide ligands as they are more rigid and have internal symmetry axes that can be aligned with crystal lattice symmetry axes, and hence we reasoned that materials generated using them should be more programmable. We aimed to design specific MOF lattices using geometrically compatible symmetric peptides and metal sites, hypothesizing that the cyclic peptides would maintain their backbone conformation and that metal binding side chains would fully coordinate metals in predictable geometries.

### Results

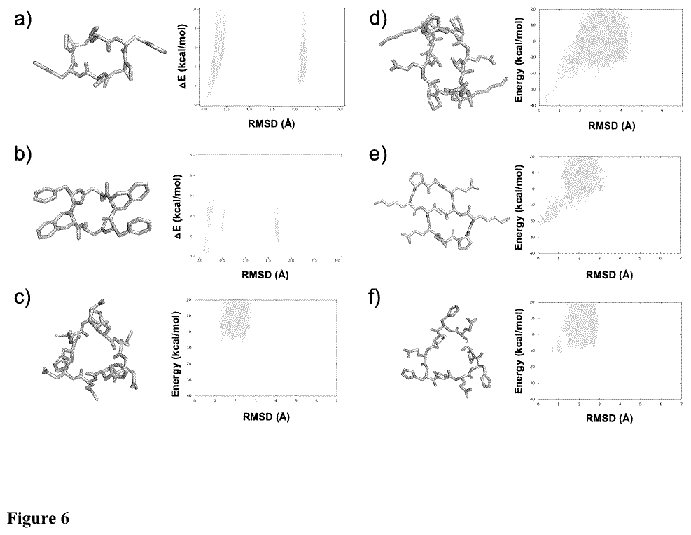

The method starts by generating large numbers of cyclic peptide backbones with internal symmetry, search for low energy sequences for these backbones, and then check by folding simulations that the lowest energy conformation matches the designed conformation. In our previous work, we designed large numbers of such compounds in silico. We were able to solve crystal structures of 12 of them that were very close to the design models, including one peptide designed to switch from one conformation into another in the presence of zinc (both conformations were confirmed crystallographically).²⁵ To generate coordination polymers using these rigid symmetric structures as building blocks, we incorporated metal liganding amino acid side chains into the structures, confirming by in silico energy landscape mapping that the lowest energy predicted states were not affected by the amino acid substitutions (FIG. 6).

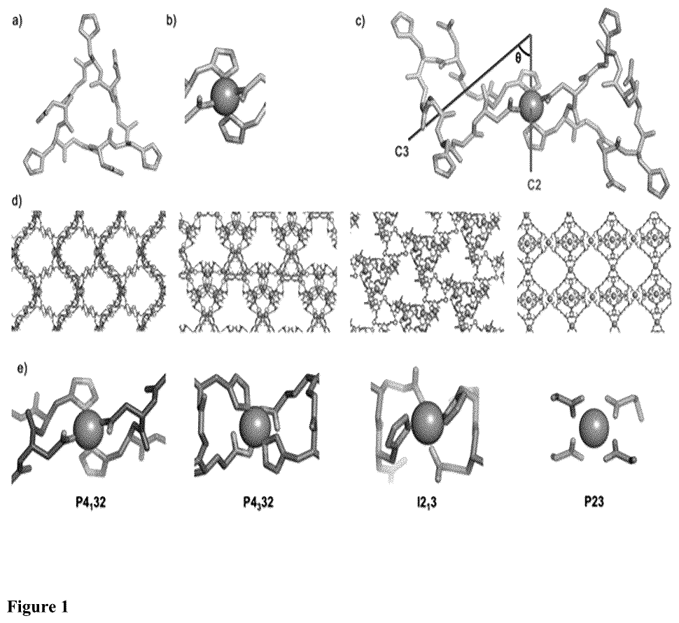

We developed a computational method for docking and designing such symmetric cyclic peptides into crystal lattices with metal mediated interfaces based on three simplifying assumptions: first, that the internal structures of the peptides would be maintained in the metal mediated crystal lattices, second that the peptides would fully coordinate metals with preferred tetrahedral geometry such as Zn²⁺ ions, and third that all metal coordinating residues would be involved in the metal coordination (for example, that peptides with one histidine and one aspartate residue would coordinate the metal in a two-His, two-Asp configuration, FIG. 1a-b). We take a bottom-up approach, starting with symmetric peptides and searching through possible interaction geometries through symmetric metal coordination sites. The peptide and metal symmetry elements are placed relative to each other based on the coordinating residue position and rotamer, as well as the metal-residue bond. To form a 3D crystal, the axes of component symmetry elements must be placed at precise dihedral angles (FIG. 1). In cases where the peptide to metal connection is a single residue, the metal-residue bond can be rotated by intersecting two cones with tips at the origin, one around the C3 axis of the peptide with width equal to the required dihedral angle, the other centered on the rotatable bond axis with width equal to the angle between the bond axes and the symmetry axis of the metal coordination site (FIG. 1c). We also considered a two residue bidentate ASP-HIS binding motif, forming an overall C2 symmetric metal site around a tetrahedral metal center (FIG. 1e). ASP-HIS pairs were precomputed and indexed, then superimposed on the peptide scaffolds. In this case, there is no rotatable metal-peptide bond, so not all structures have the appropriate dihedral angle between symmetry elements, and many must be discarded. In addition to component symmetry elements forming the correct dihedral angle between their axes, they must be placed properly within the crystal unit cell, and there must not be clashes between symmetrically related copies; evaluating these properties is lattice dependent. In the case of a C3 peptide and a C3 metal center, a P2₁3 crystal can be formed with one C3 axis along [1,1,1] and intersecting the origin, the other along [1,1,−1] and intersecting the [0,1,0] axis. The cell dimension is in this case defined by the distance from the origin to the [0,1,0] intersection. In the case of a C3 peptide and a bidentate C2 binding site, an I2₁3 crystal can be formed in a similar manner. In the case of a C3 peptide and a tetrahedral metal site, the fully coordinated site has D2 local symmetry and can form a P23 crystal by placing the D2 element axis-aligned with center along the [1,1,0] axis. The C3 element is aligned to [1,1,1], and the system scales such that the C3 axis intersects the [−2,1,1] axis. In this third case, the placement of the D2 and C3 elements each imply a unit cell dimension, and only systems where these cell dimensions agree are valid. This pipeline produced models in the P4₃32, P4₁32, P23, and I2₁3 space groups (FIG. 1d-e) which were designed using Rosetta™ (supplemental methods). Designed lattices with very low energies (as computed by Rosetta™), cell dimensions less than 50 Å, and approximate solvent fraction less than 0.8 were selected for further analysis.

To increase the diversity of structures that could be generated, we included as potential building blocks a larger in silico set of designs predicted to adopt low energy symmetric states. We selected 48 C3 peptide crystals generated from these compounds in the I2₁3, P23, P4₁32, and P4₃32 space groups with Zn²⁺ as a metal ligand for crystal assembly. The cyclic peptide ligands were synthesized in-house using previously described methods or obtained from WuXi AppTec.²⁵ To sample a wide condition space for crystallization and reduce the mass of peptide required for each individual reaction, we performed high throughput screening experiments in 5 uL of volume using 96-well plates. In a typical experiment, 1 to 2.5 mM peptide was mixed with a metal source (e.g. Zn(NO₃)₂, Fe(NO₃)₃, Cu(NO₃)₂, or Co(NO₃)₂) at various molar ratios, in the presence of aqueous buffer solution (HEPES pH 7.0-8.5 or IVIES pH 5.0-7.0), or mixtures of organic solvents (DMF, DEF, MeOH, EtOH, and/or ACN) (Tables S1 and S2). The reaction mixtures were sealed and reacted for 24-48 hours at either room temperature or at an elevated temperature (e.g. 65° C. or 80° C.) in a convection oven.

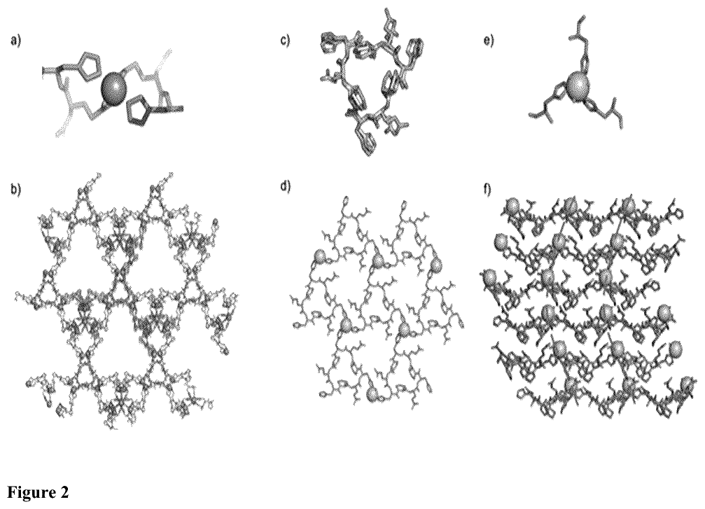

Crystallization studies reveal that many of the designed peptides formed aggregates in the presence of metals and two crystallized but could not be solved due to their low resolution (Data not shown). We were able to solve the structure of one peptide C3-1 (EhPEhPEhP; (SEQ ID NO:1)) which in the designed crystal lattice (P4₃32 space group) was intended to coordinate tetrahedral metals such as zinc with histidines and glutamates (FIG. 2a-b). We were unable to crystallize the peptide using Zn(OAc)₂, Zn(NO₃)₂, or ZnCl₂, but in the presence of Co(NO₃)₂ in HEPES pH 8.2, crystals grew in the P65 space group over 4 weeks at room temperature (FIG. 2df), and we were able to solve the structure at 0.86 Å resolution. The peptide backbone conformation matches the design with Cα RMSD of 0.59 Å (FIG. 2c). However, in the design model the metal ion is fully coordinated by the glutamic acids and histidines, while in the crystal structure each Co²⁺ cation is octahedrally coordinated to three water molecules and three histidines from different peptides in a planar fashion (FIG. 2e), and the glutamates do not participate in coordination but fill the crystal pores. This coordination geometry leads to the formation of 2D planes with 3-fold symmetry (FIG. 2d) which stack at a 60 degree offset angle (FIG. 2f) along the c-axis to form a 6-layer repeat unit (FIG. 2f, dashed lines). The 3D lattice is stabilized by dispersion interactions and hydrogen bonding between the peptide planes and is more dense than the design model (void volume of 40% compared to 91%).³⁰ Thus, while the internal conformation of the peptide matches the design model, the interactions between peptides are quite different than in the design model, with favorable peptide-peptide interactions outweighing the energetic gain from full metal coordination. These results suggest our assumption that lowest energy states would involve full metal coordination may not hold generally.

To gain further insight into the balance between peptide-peptide and peptide-metal interactions in determining MOF structures, we carried out a bottom-up exploration of peptides with variable symmetries (C2, C3, and S2), incorporated non-canonical metal coordinating residues (3-(4-Pyridyl)-alanine, DOPA, or 4-Carboxy-phenylalanine), and generated five additional structures which we describe in the following sections.

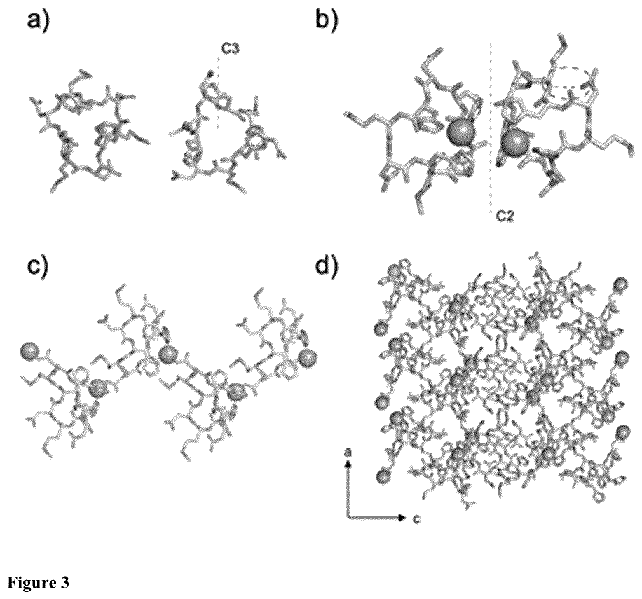

A nine residue peptide (DhmDhmDhm, (SEQ ID NO:2) C3-2, FIG. 3a,), crystallized in the P4₁2₁2 space group in the presence of 1 equivalent of Zn(NO₃)₂ in IVIES pH 6, at 80° C. for 24 hours. In contrast to the C3-1 crystal, in which the peptide conformation was nearly identical to the design model (FIG. 3a), the C3-2 peptide conformation in the metal mediated crystal is different from the original design model. This is due to a change in the torsional angle of the coordinating histidine (FIG. 3a), such metal induced changes have been observed previously.^{25,31} The backbone conformation is still C3 symmetric, but the side chain rotamers are not symmetric. The zinc ion is internally coordinated with three histidines from one peptide and an aspartic acid from an adjacent peptide (FIG. 3c). The crystal is composed of 1D metal mediated peptide chains that intercross to form a dense 3D lattice (18% calculated void volume). In the crystal, two peptide-metal chains are intertwined via dispersion interactions (FIG. 3d), and the other uncoordinated aspartic acid side chains form polar interactions with the peptide backbones (FIG. 3b, dashed circle).

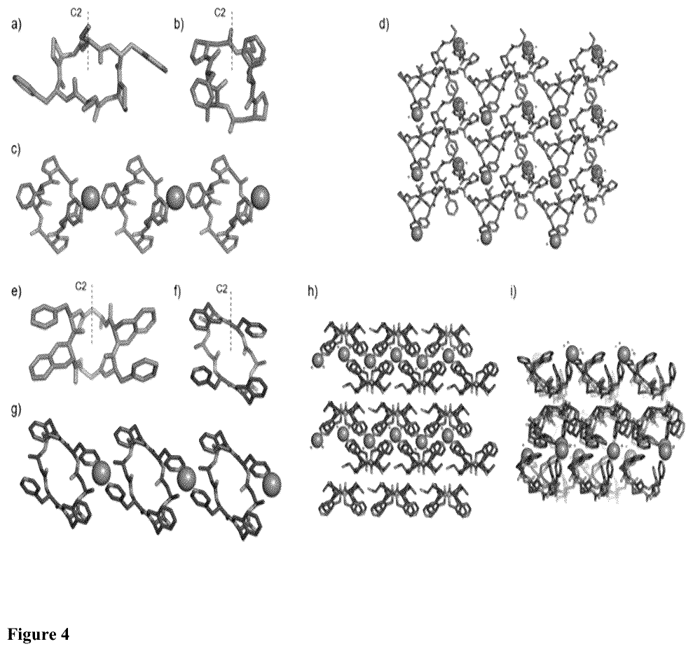

To reduce the chance of backbone conformational changes, and to explore a broader range of geometries and metal coordination ligands, we used a geometric hashing approach to design two pyridine-containing 6-mer peptides with AIMNet ground states having C2 symmetry (FIG. 6a-b, methods), and were able to obtain crystals with metal in multiple conditions after heating at 80° C. for two days. The structures of the crystals formed with 1 equivalent Zn(NO₃)₂ are shown in FIG. 4. C2-1 (FIG. 4a) formed crystals in the presence of HEPES pH 7.5 in the P1 (FIG. 4a-d) space group, and C2-2 (FIG. 4e) formed crystals in both the C121/P12₁1 space groups (FIG. 4e-i). In both cases, crystallization was driven by Zn-pyridine interactions which formed 1D metal-peptide chains (FIG. 4c and FIG. 4g) that hierarchically thread into 3D crystals.

In the lattice formed by the C2-1 ligand (3-(4-Pyridyl)-alanine-β-Homoproline-α-Aminobutyric acid-3-(4-Pyridyl)-alanine-β-Homoproline-α-Aminobutyric acid) (SEQ ID NO:3) each zinc ion is linked to two peptides through pyridine coordination while two water molecules fill the other positions for full tetrahedral coordination (FIG. 4c). The resulting peptide chains form 3D crystals through peptide stacking that is mediated by dispersion interactions and hydrogen bonding with participating water molecules. The 1D metal-peptide coordination chains grow along two different directions and intersect with each other, tiling the ab plane (FIG. 4d); non-covalent interactions mediate the stacking of these layers into 3D crystals. Water filled pores between the coordination chains make up 25% of the calculated unit cell volume (SI mercury). The internal hydrogen bonds in the peptide design model are broken in the crystal; AIMNet calculates the crystal conformation to be 4.7 kcal/mol higher in energy than the designed conformation suggesting that the lattice stabilizes the higher energy state.

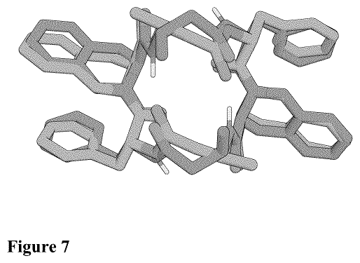

The crystal structure of peptide C2-2 (3-(4-Pyridyl)-alanine-1,2,3,4-tetrahydroisoquinoline-3-carboxylic acid-3-Aminobutanoic acid-3-(4-Pyridyl)-alanine-1,2,3,4-tetrahydroisoquinoline-3-carboxylic acid-3-Aminobutanoic acid) in the absence of metal in methanol matches that of the design model (FIG. 7). Crystal structures in the presence of Zn²⁺ reveal a different peptide conformation 3.4 kcal/mol higher than the designed conformation according to AIMNet (FIG. 4e-f). The first crystal (P12₁1 space group, FIG. 4h) formed in HEPES pH 8.0 and 2% PEG2000 has a void volume of 35.3%. The second crystal (C121 space group, FIG. 4i) formed in HEPES pH 8.0 and 2% PEP and has a void volume of 16%. As in the C2-1 case, in both crystals the Zn²⁺ ions are tetrahedrally coordinated with two pyridine ligands and two water molecules and form 1D chains (FIG. 4g), but the packing is slightly different. For the C121 crystal, 1D chains first arrange in a parallel fashion into bilayer planes, which then stack to form the 3D crystal lattice (FIG. 4i). The P12₁1 crystal shares an identical peptide-zinc coordination configuration, but the 1D chains stack with different angles between the adjacent 2D planes. Thus, for this peptide, 1D coordination chains are mediated by metal coordination, and higher order structures such as 2D planes and 3D crystals are stabilized by dispersion interactions through extensive interchain peptide-peptide packing.

We next explored metal mediated crystals built from achiral S2 symmetric peptides. These peptides have a two-fold improper rotation across their axis of symmetry allowing access to centrosymmetric space groups which increases the likelihood of crystallization.³³ Crystal structures determined in the absence of metal are very close to the design models.²⁵

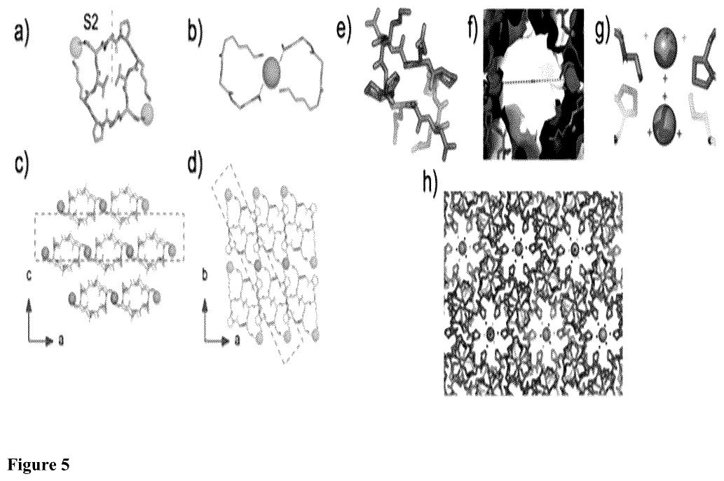

S2-1(ppKvEPPkVe) (SEQ ID NO:6), is a 10 residue S2 symmetric cyclic peptide containing one lysine and one glutamate per asymmetric unit (FIG. 5a). The apo structure matches the design to 0.53 Å RMSD. S2-1 formed crystals upon heating at 80° C. for 24 hours in DMF with one equivalent of Cu(NO₃)₂ in the P-1 space group (FIG. 5c-d) with very small pores making up 7% of the unit cell volume. A single Cu²⁺ is coordinated between two peptides via two lysines and two glutamates in a square planar geometry (FIG. 5b). Each peptide forms a bidentate interaction with two Cu²⁺ ions that assemble into a crystal through peptide backbone hydrogen bonding (FIG. 5c-d). Despite the copper coordination, the peptide backbone conformation matches that of the design and the apo structure. The crystal lattice also matches that of the apo crystal with an expansion of the a and b axis by 1 Å each to allow for metal incorporation into the structure.²⁵

The 12 residue S2-2 peptide (aNkhPeAnKHpE (SEQ ID NO:5), FIG. 5e) contains one lysine, one histidine, and one glutamic acid per asymmetric unit available for metal coordination. The apo structure of this peptide matches the design to 0.43 Å RMSD.²⁵ In the presence of 1 equivalent of ZnCl₂, S2-2 crystallizes in isopropanol at room temperature in the R3 space group (FIG. 5f). In the crystal structure, peptide-peptide interactions mediate crystal packing, while the large open channels along the c axis make up 40% of the unit cell volume (FIG. 5f), which are filled with water-coordinated Zn²⁺ ions (FIG. 5g). Comparison of the structure of S2-2 obtained in the absence and presence of metals indicate that the addition of Zn²⁺ did not change the overall crystal packing, since the Zn²⁺ ions occupied empty open channels in the crystal and are not coordinated to any of the aforementioned metal-binding residues (FIG. 5g). The peptide-peptide interactions in this crystal lattice are evidently more favorable than the metal coordination in the crystal conditions screened.

### CONCLUSIONS

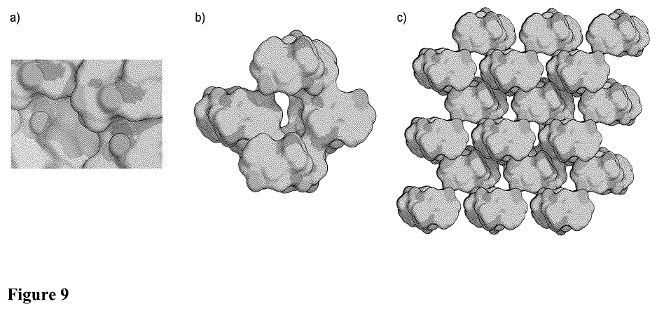

MOFs comprising large peptide ligands with internal symmetry have not to our knowledge been previously explored. We report the first structures of symmetric cyclic 6 to 12 residue peptide MOFs with both proper and improper symmetries (C2, C3, and S2), employing for metal-chelation histidine, cysteine, aspartate, glutamate, and noncanonical amino acids containing pyridine and DOPA side chains. Our crystal structures of six peptide MOFs with different metals (Zn²⁺, Co²⁺, and Cu²⁺) and space groups (P1, P65, C121, P12₁1, R3, P4₁2₁2, and P-1) contain a rich variety of 1D and 2D metal-mediated structures with pore shapes and sizes ranging from 7% to 40% void volume (some of these features have been observed in previous peptide-metal crystal structures, for example, six residue poly-proline peptides can assemble into strings mediated by zinc and form dense frameworks through proline-proline packing).¹⁷ The up to 12 residue cyclic peptide ligands studied here are to our knowledge the largest peptidic ligands reported that form crystalline coordination polymers to date. An essentially unlimited number of rigid symmetric cyclic peptides can be designed using the methods described in Mulligan et al.²⁵, and hence the crystal lattices described here are the first representatives of a very large class of new metal-organic crystals that could provide new peptide materials for biocompatible, chiral, and catalytic applications. The large surface area and pore sizes of these peptide-metal lattices make them particularly interesting for downstream applications such as catalysis and sensing, and the wide variety of both natural and unnatural sidechains available allows facile customization of chemistry lining the pores and other structural features of the crystals. The lattices frequently contain open metal coordination sites (FIG. 9), around which substrate binding pockets could be built by further computational design, providing access to a new class of catalytic materials combining features of MOFs and enzymes.

### REFERENCES

(1) Yaghi, O. M.; O'Keeffe, M.; Ockwig, N. W.; Chae, H. K.; Eddaoudi, M.; Kim, J. Reticular Synthesis and the Design of New Materials. Nature 2003, 423 (6941), 705-714.
(2) Furukawa, H.; Cordova, K. E.; O'Keeffe, M.; Yaghi, 0. M. The Chemistry and Applications of Metal-Organic Frameworks. Science 2013, 341 (6149), 1230444.
(3) Ji, Z.; Wang, H.; Canossa, S.; Wuttke, S.; Yaghi, O. M. Pore Chemistry of Metal-organic Frameworks. Adv. Funct. Mater. 2020, 30 (41), 2000238.
(4) Zhou, H.-C. “joe”; Kitagawa, S. Metal-Organic Frameworks (MOFs). Chem. Soc. Rev. 2014, 43 (16), 5415-5418.
(5) Bailey, J. B.; Tezcan, F. A. Tunable and Cooperative Thermomechanical Properties of Protein-Metal-Organic Frameworks. J. Am. Chem. Soc. 2020, 142 (41), 17265-17270.
(6) Chiong, J. A.; Zhu, J.; Bailey, J. B.; Kalaj, M.; Subramanian, R. H.; Xu, W.; Cohen, S. M.; Tezcan, F. A. An Exceptionally Stable Metal-Organic Framework Constructed from Chelate-Based Metal-Organic Polyhedra. J. Am. Chem. Soc. 2020, 142 (15), 6907-6912.
(7) Keskin, S.; Kizilel, S. Biomedical Applications of Metal Organic Frameworks. Ind. Eng. Chem. Res. 2011, 50 (4), 1799-1812.
(8) Li, J.-R.; Sculley, J.; Zhou, H.-C. Metal-Organic Frameworks for Separations. Chem. Rev. 2012, 112 (2), 869-932.
(9) Baek, J.; Rungtaweevoranit, B.; Pei, X.; Park, M.; Fakra, S. C.; Liu, Y-S.; Matheu, R.; Alshmimri, S. A.; Alshehri, S.; Trickett, C. A.; Somorjai, G. A.; Yaghi, O. M. Bioinspired Metal-Organic Framework Catalysts for Selective Methane Oxidation to Methanol. J. Am. Chem. Soc. 2018, 140 (51), 18208-18216.
(10) Kreno, L. E.; Leong, K.; Farha, O. K.; Allendorf, M.; Van Duyne, R. P.; Hupp, J. T. Metal-Organic Framework Materials as Chemical Sensors. Chem. Rev. 2012, 112 (2), 1105-1125.
(11) Lee, J.; Farha, O. K.; Roberts, J.; Scheidt, K. A.; Nguyen, S. T.; Hupp, J. T. Metal-Organic Framework Materials as Catalysts. Chem. Soc. Rev. 2009, 38 (5), 1450-1459.
(12) Huxford, R. C.; Della Rocca, J.; Lin, W. Metal-organic Frameworks as Potential Drug Carriers. Curr. Opin. Chem. Biol. 2010, 14 (2), 262-268.
(13) Xiao, D. J.; Oktawiec, J.; Milner, P. J.; Long, J. R. Pore Environment Effects on Catalytic Cyclohexane Oxidation in Expanded Fe2(dobdc) Analogues. J. Am. Chem. Soc. 2016, 138 (43), 14371-14379.
(14) Chui, S. S.-Y.; Lo, S. M.-F.; Charmant, J. P. H.; Orpen, A. G.; Williams, I. D. A Chemically Functionalizable Nanoporous Material [Cu 3 (TMA) 2 (H2O) 3] N. Science 1999,283 (5405), 1148-1150.
(15) Deng, H.; Grunder, S.; Cordova, K. E.; Valente, C.; Furukawa, H.; Hmadeh, M.; Gándara, F.; Whalley, A. C.; Liu, Z.; Asahina, S.; Kazumori, H.; O'Keeffe, M.; Terasaki, O.; Stoddart, J. F.; Yaghi, O. M. Large-Pore Apertures in a Series of Metal-Organic Frameworks. Science 2012, 336 (6084), 1018-1023.
(16) Dong, J.; Liu, Y.; Cui, Y. Artificial Metal-Peptide Assemblies: Bioinspired Assembly of Peptides and Metals through Space and across Length Scales. J. Am. Chem. Soc. 2021,143 (42), 17316-17336.
(17) Schnitzer, T.; Paenurk, E.; Trapp, N.; Gershoni-Poranne, R.; Wennemers, H. Peptide-Metal Frameworks with Metal Strings Guided by Dispersion Interactions. J. Am. Chem. Soc. 2021,143 (2), 644-648.
(18) Rabone, J.; Yue, Y.-F.; Chong, S. Y.; Stylianou, K. C.; Bacsa, J.; Bradshaw, D.; Darling, G. R.; Berry, N. G.; Khimyak, Y. Z.; Ganin, A. Y.; Wiper, P.; Claridge, J. B.; Rosseinsky, M. J. An Adaptable Peptide-Based Porous Material. Science 2010,329 (5995), 1053-1057.
(19) Chino, M.; Maglio, O.; Nastri, F.; Pavone, V.; DeGrado, W. F.; Lombardi, A. Artificial Diiron Enzymes with a DE Novo Designed Four-Helix Bundle Structure. Eur. J. Inorg. Chem. 2015, 2015 (21), 3371-3390.
(20) Kim, J. D.; Pike, D. H.; Tyryshkin, A. M.; Swapna, G. V. T.; Raanan, H.; Montelione, G. T.; Nanda, V.; Falkowski, P. G. Minimal Heterochiral de Novo Designed 4Fe-4S Binding Peptide Capable of Robust Electron Transfer. J. Am. Chem. Soc. 2018,140 (36), 11210-11213.
(21) Shi, J.; Li, J.; Zeng, H.; Zou, G.; Zhang, Q.; Lin, Z. Water Stable Oxalate-Based Coordination Polymers with in Situ Generated Cyclic Dipeptides Showing High Proton Conductivity. Dalton Trans. 2018, 47 (43), 15288-15292.
(22) Navarro-Sanchez, J.; Argente-Garcia, A. I.; Moliner-Martínez, Y.; Roca-Sanjuán, D.; Antypov, D.; Campins-Falcó, P.; Rosseinsky, M. J.; Martí-Gastaldo, C. Peptide Metal-Organic Frameworks for Enantioselective Separation of Chiral Drugs. J. Am. Chem. Soc. 2017,139 (12), 4294-4297.
(23) Katsoulidis, A. P.; Antypov, D.; Whitehead, G. F. S.; Carrington, E. J.; Adams, D. J.; Berry, N. G.; Darling, G. R.; Dyer, M. S.; Rosseinsky, M. J. Chemical Control of Structure and Guest Uptake by a Conformationally Mobile Porous Material. Nature 2019, 565 (7738), 213-217.
(24) Meng, W.; Kondo, S.; Ito, T.; Komatsu, K.; Pirillo, J.; Hijikata, Y.; Ikuhara, Y; Aida, T.; Sato, H. An Elastic Metal-organic Crystal with a Densely Catenated Backbone. Nature 2021, 598 (7880), 298-303.
(25) Mulligan, V. K.; Kang, C. S.; Sawaya, M. R.; Rettie, S.; Li, X.; Antselovich, I.; Craven, T. W.; Watkins, A. M.; Labonte, J. W.; DiMaio, F.; Yeates, T. O.; Baker, D. Computational Design of Mixed Chirality Peptide Macrocycles with Internal Symmetry. Protein Sci. 2020, 29 (12), 2433-2445.
(26) Hosseinzadeh, P.; Bhardwaj, G.; Mulligan, V. K.; Shortridge, M. D.; Craven, T. W.; Pardo-Avila, F.; Rettie, S. A.; Kim, D. E.; Silva, D.-A.; Ibrahim, Y. M.; Webb, I. K.; Cort, J. R.; Adkins, J. N.; Varani, G.; Baker, D. Comprehensive Computational Design of Ordered Peptide Macrocycles. Science 2017, 358 (6369), 1461-1466.
(27) Leman, J. K.; Weitzner, B. D.; Lewis, S. M.; Adolf-Bryfogle, J.; Alam, N.; Alford, R. F.; Aprahamian, M.; Baker, D.; Barlow, K. A.; Barth, P.; Basanta, B.; Bender, B. J.; Blacklock, K.; Bonet, J.; Boyken, S. E.; Bradley, P.; Bystroff, C.; Conway, P.; Cooper, S.; Correia, B. E.; Coventry, B.; Das, R.; De Jong, R. M.; DiMaio, F.; Dsilva, L.; Dunbrack, R.; Ford, A. S.; Frenz, B.; Fu, D. Y.; Geniesse, C.; Goldschmidt, L.; Gowthaman, R.; Gray, J. J.; Gront, D.; Guffy, S.; Horowitz, S.; Huang, P.-S.; Huber, T.; Jacobs, T. M.; Jeliazkov, J. R.; Johnson, D. K.; Kappel, K.; Karanicolas, J.; Khakzad, H.; Khar, K. R.; Khare, S. D.; Khatib, F.; Khramushin, A.; King, I. C.; Kleffner, R.; Koepnick, B.; Kortemme, T.; Kuenze, G.; Kuhlman, B.; Kuroda, D.; Labonte, J. W.; Lai, J. K.; Lapidoth, G.; Leaver-Fay, A.; Lindert, S.; Linsky, T.; London, N.; Lubin, J. H.; Lyskov, S.; Maguire, J.; Malmström, L.; Marcos, E.; Marcu, O.; Marze, N. A.; Meiler, J.; Moretti, R.; Mulligan, V. K.; Nerli, S.; Norn, C.; Ó'Conchúir, S.; Ollikainen, N.; Ovchinnikov, S.; Pacella, M. S.; Pan, X.; Park, H.; Pavlovicz, R. E.; Pethe, M.; Pierce, B. G.; Pilla, K. B.; Raveh, B.; Renfrew, P. D.; Burman, S. S. R.; Rubenstein, A.; Sauer, M. F.; Scheck, A.; Schief, W.; Schueler-Furman, O.; Sedan, Y; Sevy, A. M.; Sgourakis, N. G.; Shi, L.; Siegel, J. B.; Silva, D.-A.; Smith, S.; Song, Y.; Stein, A.; Szegedy, M.; Teets, F. D.; Thyme, S. B.; Wang, R. Y-R.; Watkins, A.; Zimmerman, L.; Bonneau, R. Macromolecular Modeling and Design in Rosetta: Recent Methods and Frameworks. Nat. Methods 2020, 17 (7), 665-680.
(28) Alford, R. F.; Leaver-Fay, A.; Jeliazkov, J. R.; O'Meara, M. J.; DiMaio, F. P.; Park, H.; Shapovalov, M. V.; Renfrew, P. D.; Mulligan, V. K.; Kappel, K.; Labonte, J. W.; Pacella, M. S.; Bonneau, R.; Bradley, P.; Dunbrack, R. L., Jr; Das, R.; Baker, D.; Kuhlman, B.; Kortemme, T.; Gray, J. J. The Rosetta All-Atom Energy Function for Macromolecular Modeling and Design. J. Chem. Theory Comput. 2017, 13 (6), 3031-3048.
(29) Holm, R. H.; Kennepohl, P.; Solomon, E. I. Structural and Functional Aspects of Metal Sites in Biology. Chem. Rev. 1996, 96 (7), 2239-2314.
(30) Macrae, C. F.; Sovago, I.; Cottrell, S. J.; Galek, P. T. A.; McCabe, P.; Pidcock, E.; Platings, M.; Shields, G. P.; Stevens, J. S.; Towler, M.; Wood, P. A. Mercury 4.0: From Visualization to Analysis, Design and Prediction. Journal of Applied Crystallography. 2020, pp 226-235. doi.org/10.1107/s1600576719014092.
(31) Párraga, G.; Horvath, S. J.; Eisen, A.; Taylor, W. E.; Hood, L.; Young, E. T.; Klevit, R. E. Zinc-Dependent Structure of a Single-Finger Domain of Yeast ADR1. Science 1988, 241 (4872), 1489-1492.
(32) Zubatyuk, R.; Smith, J. S.; Leszczynski, J.; Isayev, O. Accurate and Transferable Multitask Prediction of Chemical Properties with an Atoms-in-Molecules Neural Network. Sci Adv 2019, 5 (8), eaav6490.
(33) Yeates, T. O.; Kent, S. B. H. Racemic Protein Crystallography. Annu. Rev. Biophys. 2012, 41, 41-61.
(34) Dou, J.; Vorobieva, A. A.; Sheffler, W.; Doyle, L. A.; Park, H.; Bick, M. J.; Mao, B.; Foight, G. W.; Lee, M. Y; Gagnon, L. A.; Carter, L.; Sankaran, B.; Ovchinnikov, S.; Marcos, E.; Huang, P.-S.; Vaughan, J. C.; Stoddard, B. L.; Baker, D. De Novo Design of a Fluorescence-Activating β-Barrel. Nature 2018, 561 (7724), 485-491.

### Computational Methods

Peptide generation: C3 and S2 symmetric macrocycles backbones were generated using the Rosetta™ protocol. The C3 scaffolds were then matched into 3D lattices using the protocol described below. Peptide sequences resulting from the 3D lattice matching were filtered based on their ability to favor the designed monomeric conformation. We used the same protocol described n Mulligan et al. using the Simple_cycpep_predict application to sample conformations of each C3 and S2 peptide. Then we calculated the Rosetta™ energy for each conformation and plotted the calculated energy against backbone RMSD to the designed conformation.

C2 symmetric macrocycles were generated using a geometric hashing protocol. Low energy conformers of monomeric building blocks were generated, and the rigid body transformations associated with these conformations were computed. Next, all rigid body transforms of N-mers generated by linking N monomers were computed using simple matrix multiplication of the outer-product of the chosen monomers' conformers. N-mer conformers that result in C2-symmetric 2N-mers were identified by calculating the angle of rotation and translation about the rotation axis of the transforms. This angle must equal 180 degrees while the translation must be zero to satisfy C2 symmetry. Full-atom representations of the resulting combinations of the chosen monomers' conformers that satisfy these criteria were built and minimized with AIMNet.

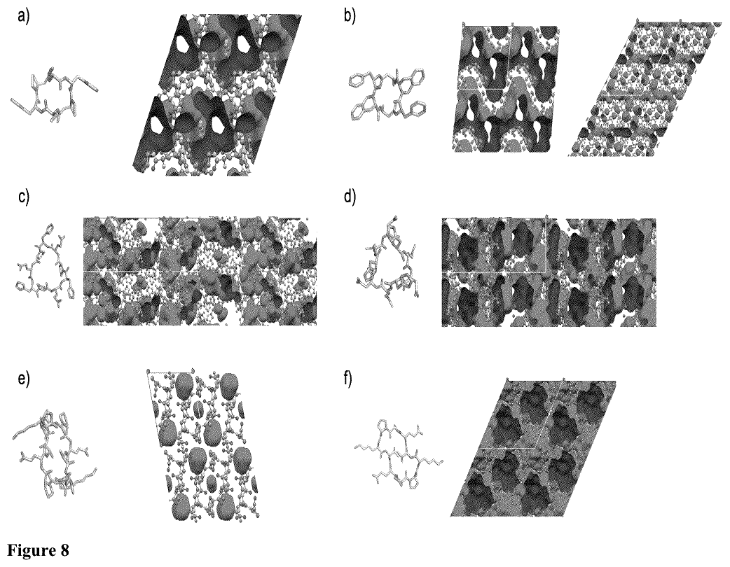

Metal mediated crystal lattice design: Our design approach is similar in principle to that of King et al. and Hsia et al., wherein distinct symmetry elements are placed so they propagate into a desired assembly. A top-down approach was used in King et al., placing proteins with cyclic symmetry along the axes of the target cage symmetry, for example C4 and C3 at the faces and corners of a cube, then sampling the rotations and translations along these axes that preserve symmetry.³ A bottom-up approach was used in Hsia et al., fusing proteins with cyclic symmetry through helical repeat linker elements and searching for fusions which place the symmetry elements relative to each other to form a target symmetry, for example forming a cube with C4 and C3 elements 54.7 degrees apart such that the axes intersect.⁴ The bottom up approach we use here to design crystal lattices starting with symmetric peptides and searching possible binding geometries that attach a symmetric metal coordination site goes beyond the previous approaches in several ways. First, here the relationship between symmetry elements is defined by rotamer and metal binding geometry rather than protein-protein interactions or backbone backbone fusion. Second, we design three dimensional crystal assemblies requiring more complex geometric criteria, precision, and careful alignment to the unit cell. Third, we employ small peptide scaffolds with D and L amino acids rather than large all L proteins. Fourth, we considered D2 symmetry elements as well as cyclic elements. Consideration of D amino acids and D2 symmetry elements expands the space of possible symmetric assemblies and metal binding geometries, but is otherwise straightforward. Placement of symmetry elements to form 3D crystals requires higher precision than in other symmetric design tasks, as small errors can propagate much further in the assembly before self-reinforcement. For example, C4 elements on the faces of a cube require only three steps to come back on itself, while a P2₁3 crystal requires ten steps. To ameliorate this issue, we sample metal binding rotamers in 1 degree steps through a set of python packages to dock and design cyclic peptides into four crystal lattice space groups. Crystal properties: To calculate the void volume in each crystal structure, water was removed from the structures (FIG. 8), then the percent void in a unit cell was calculated using Mercury's default settings as described in Macrae et al.

### Materials and Methods

Peptide synthesis: All peptides were purchased from WuXi Apptec or synthesized in-house on a microwave synthesizer. All L and D amino acids were purchased from P3 Biosystems. Oxyma Pure™ was purchased from CEM, DIC was purchased from Oakwood Chemical, diisopropyl ethylamine (DIEA) and piperidine were purchased from Sigma Aldrich. DMF was purchased from Fisher Scientific and treated with an Aldraamine trapping pack prior to use. Synthesis was done on a 0.1 mmol scale on CEM Cl-TCP(Cl) resin. Five equivalents of each amino acid were activated using 0.1M Oxyma with 2% (v/v) DIEA in DMF, 15.4% (v/v) DIC, and coupled on resin for 4 minutes with double coupling if needed. This was followed by deprotection using 5 mL of 20% piperidine in Dimethylformamide (DMF) for 2 minutes at 95° C. Completed linear peptides were removed from resin while maintaining side chain protecting groups by 5 times 5 minute incubations of the resin in 1% TFA in DCM. The DCM was removed in vacuo and the protected peptides were subjected to lyophilization in a 1:1 water:ACN mixture. The protected peptides were resuspended in 70 mL DCM in a 100 mL round bottom flask, treated with 1.1 equivalents (7-Azabenzotriazol-1-yloxy)tripyrrolidinophosphonium hexafluorophosphate (PyAOP), and stirred for 30 minutes before adding 0.2% (v/v) DIEA dropwise. The cyclization reaction proceeded for 16 hours before removing DCM in vacuo and subjecting the peptide to a total deprotection solution consisting of TFA/H2O/DODT/TIPS (92.5:2.5:2.5:2.5) for 3 hours. This deprotection mixture was precipitated in 30 mL ice cold ethyl ether, centrifuged and decanted, then washed twice more with fresh ether and dried under nitrogen to yield crude peptide for HPLC purification.

Peptide Purification: The crude peptide was dried and dissolved in a mixture of acetonitrile (ACN) and water where the entire crude is soluble. This solution was purified on a C18 column in an Agilent high pressure liquid chromatography (HPLC) instrument. A linear gradient of increasing ACN with 0.1% TFA was used to purify the samples. UV signal was monitored at 214 nm and all peaks were collected. Peaks were checked using ESI mass spectroscopy for the correct peptide mass. The purified peptide was then lyophilized for further use. All UPLC and mass spectra are included in the supplementary data.

Crystal screening: Peptides were screened using 96 well plates using the conditions shown in supplementary tables 1 and 2. Stocks of the peptides were made in water, methanol, acetonitrile, or DMF so that 1.25-5 mM are added to each well. The peptide samples were left to dry in the plate overnight then 5 uL of the appropriate solvent was added to each well. Completed plates were incubated at 4° C. overnight then checked using a light microscope for crystal formation. If no crystals form, the plates were placed in a convection oven at 80° C. Once crystals formed, diffraction data were collected from a single crystal at synchrotron (on APS 24ID-C) and at 100 K. Unit cell refinement, and data reduction were performed using XDS and CCP4 suites. The structure was identified by direct methods and refined by full-matrix least-squares on F2 with anisotropic displacement parameters for the non-H atoms using SHELXL-2018/3. Structure analysis was aided by using Coot/Shelxle. The hydrogen atoms on heavy atoms were calculated in ideal positions with isotropic displacement parameters set to 1.2×U_{eq} of the attached atoms. Crystallographic structures were deposited into the Cambridge Structural Database (CSD), under deposition numbers 2160569 (C2-1), 2160570 (C2-2a), 2160571 (C2-2b), 2160572 (C3-1), 2160573 (C3-2), 2160589 (S2-1), 2160766 (S2-2).

### Supplementary Data

Analytical UPLC and LCMS spectra for each peptide are shown in Table 1. Percent purity is calculated based on area integration of the analytical plot.

TABLE 1

| | Mass detected | Percent Purity |
|---|---|---|
| Peptide | by LCMS (M/Z) | detected by UPLC |
| C2-1 | 689.27 | 97% |
| C2-2 | 785.35 | 94% |
| C3-1 | 1090.40 | 96% |
| C3-2 | 1150.53 | 95% |
| S2-1 | 1101.59 | 92% |
| S2-2 | 1353.80 | 97% |

### REFERENCES

(1) Mulligan, V. K.; Kang, C. S.; Sawaya, M. R.; Rettie, S.; Li, X.; Antselovich, I.; Craven, T. W.; Watkins, A. M.; Labonte, J. W.; DiMaio, F.; Yeates, T. O.; Baker, D. Computational Design of Mixed Chirality Peptide Macrocycles with Internal Symmetry. Protein Sci. 2020, 29 (12), 2433-2445.
(2) Zubatyuk, R.; Smith, J. S.; Leszczynski, J.; Isayev, O. Accurate and Transferable Multitask Prediction of Chemical Properties with an Atoms-in-Molecules Neural Network. Sci Adv 2019, 5 (8), eaav6490.
(3) King, N. P.; Bale, J. B.; Sheffler, W.; McNamara, D. E.; Gonen, S.; Gonen, T.; Yeates, T. O.; Baker, D. Accurate Design of Co-Assembling Multi-Component Protein Nanomaterials. Nature 2014, 510 (7503), 103-108.
(4) Hsia, Y.; Mout, R.; Sheffler, W.; Edman, N. I.; Vulovic, I.; Park, Y.-J.; Redler, R. L.; Bick, M. J.; Bera, A. K.; Courbet, A.; Kang, A.; Brunette, T. J.; Nattermann, U.; Tsai, E.; Saleem, A.; Chow, C. M.; Ekiert, D.; Bhabha, G.; Veesler, D.; Baker, D. Design of Multi-Scale Protein Complexes by Hierarchical Building Block Fusion. Nat. Commun. 2021, 12 (1), 2294.
(5) Macrae, C. F.; Sovago, I.; Cottrell, S. J.; Galek, P. T. A.; McCabe, P.; Pidcock, E.; Platings, M.; Shields, G. P.; Stevens, J. S.; Towler, M.; Wood, P. A. Mercury 4.0: From Visualization to Analysis, Design and Prediction. Journal of Applied Crystallography. 2020, pp 226-235. doi.org/10.1107/s1600576719014092.
(6) Kabsch, W. XDS. Acta Crystallogr. D Biol. Crystallogr. 2010, 66 (Pt 2), 125-132.
(7) Winn, M. D.; Ballard, C. C.; Cowtan, K. D.; Dodson, E. J.; Emsley, P.; Evans, P. R.; Keegan, R. M.; Krissinel, E. B.; Leslie, A. G. W.; McCoy, A.; McNicholas, S. J.; Murshudov, G. N.; Pannu, N. S.; Potterton, E. A.; Powell, H. R.; Read, R. J.; Vagin, A.; Wilson, K. S. Overview of the CCP4 Suite and Current Developments. Acta Crystallogr. D Biol. Crystallogr. 2011, 67 (Pt 4), 235-242.
(8) Sheldrick, G. M. SHELXT—Integrated Space-Group and Crystal-Structure Determination. Acta Crystallographica Section A Foundations and Advances. 2015, pp 3-8. doi.org/10.1107/s2053273314026370.
(9) Sheldrick, G. M. Crystal Structure Refinement with SHELXL. Acta Crystallogr. B 2015, 71 (Pt 1), 3-8.
(10) Emsley, P.; Cowtan, K. Coot: Model-Building Tools for Molecular Graphics. Acta Crystallogy. D Biol. Crystallogr. 2004, 60 (Pt 12 Pt 1), 2126-2132.
(11) Hübschle, C. B.; Sheldrick, G. M.; Dittrich, B. ShelXle: A Qt Graphical User Interface for SHELXL. J. Appl. Crystallogr 2011, 44 (Pt 6), 1281-1284.

We claim:

1. A cyclic peptide, comprising a sequence selected from the group consisting of:
(a) EhPEhPEhP (SEQ ID NO:1), wherein E can be substituted with any L amino acid that is not proline,
(b) DhmDhmDhm (SEQ ID NO:2), wherein m can be any alpha D amino acid that is not proline,
(c) (3-(4-Pyridyl)-alanine-β-Homoproline-D-α-Aminobutyric acid-3-(4-Pyridyl)alanine-β-Homoproline-D-α-Aminobutyric acid), wherein D-α-Aminobutyric acid can be substituted with any D amino acid that is not proline(SEQ ID NO:3),
(d) (3-(4-Pyridyl)-alanine-1,2,3,4-tetrahydroisoquinoline-3-carboxylic acid-3-Aminobutanoic acid-3-(4-Pyridyl)-alanine-1,2,3,4-tetrahydroisoquinoline-3-carboxylic acid-3-Aminobutanoic acid wherein 3-aminobutanoic acid can be substituted to any R amino acid such as β-phenylalanine, β-alanine, or 3-Aminoisobuteric acid);
(e) aNkhPeAnKHpE (SEQ ID NO:5), or salt thereof, wherein a, k, h, e, and n can be substituted to any D amino acid that is not proline; and wherein N, A, K, H, and E can be substitutes to any L amino acid that is not proline; and (ii) a metal ion; and
(f) ppKvEPPkVe (SEQ ID NO:6), or salt thereof, wherein v can be substituted to any D amino acid that is not proline, and V can be substituted to any L amino acid that is not proline;
wherein single letter amino acid residues in upper case are L amino acids, and single letter amino acid residues in lower case are D amino acids.

2. The cyclic peptide of claim 1, wherein one or more substitutable residue on the cyclic peptide comprises an additional moiety attached via a side chain of the substitutable residue or a residue substituting for the substitutable residue.

3. The cyclic peptide of claim 1, wherein one or more substitutable residue is substituted with a lysine residue, optionally wherein one or more lysine residue is conjugated to an additional moiety.

4. A multimer comprising 2 or more copies of an identical cyclic peptide of claim 1.

5. The multimer of claim 4, further comprising a metal ion.

6. The multimer of claim 5, wherein the metal ion is selected from the group consisting of Co²⁺, Zn²⁺, Fe³⁺, and Cu²⁺.

7. A metal-organic framework (MOF), comprising:
(a) a multimer comprising (i) at least two copies of cyclic peptide EhPEhPEhP (SEQ ID NO:1) or salt thereof, wherein E can be substituted with any L amino acid that is not proline, and (ii) a metal ion;
(b) a multimer comprising (i) at least two copies of cyclic peptide DhmDhmDhm (SEQ ID NO:2), or salt thereof, wherein m can be any alpha D amino acid that is not proline, and (ii) a metal ion;
(c) a multimer comprising (i) at least two copies of cyclic peptide (3-(4-Pyridyl)-alanine-β-Homoproline-α-Aminobutyric acid-3-(4-Pyridyl)-alanine-β-Homoproline-α-Aminobutyric acid), or salt thereof, wherein D-α-Aminobutyric acid can be substituted with any D amino acid that is not proline (SEQ ID NO:3), and (ii) a metal ion;
(d) a multimer comprising (i) at least two copies of cyclic peptide (3-(4-Pyridyl)-alanine-1,2,3,4-tetrahydroisoquinoline-3-carboxylic acid-3-Aminobutanoic acid-3-(4-Pyridyl)-alanine-1,2,3,4-tetrahydroisoquinoline-3-carboxylic acid-3-Aminobutanoic acid) (SEQ ID NO:4), or salt thereof; wherein 3-aminobutanoic acid can be substituted to any R amino acid such as β-phenylalanine, β-alanine, or 3-Aminoisobuteric acid, and (ii) a metal ion;
(e) a multimer comprising (i) at least two copies of cyclic peptide aNkhPeAnKHpE (SEQ ID NO:5), or salt thereof, wherein a, k, h, e, and n can be substituted to any D amino acid that is not proline; and wherein N, A, K H, and E can be substitutes to any L amino acid that is not proline; and (ii) a metal ion; or
(f) a multimer comprising (i) at least two copies of cyclic peptide ppKvEPPkVe (SEQ ID NO:6), or salt thereof, wherein v can be substituted to any D amino acid that is not proline. V can be substituted to any L amino acid that is not proline; and (ii) a metal ion;
wherein single letter amino acid residues in upper case are L amino acids, and single letter amino acid residues in lower case are D amino acids.

8. The MOF of claim 7, wherein one or more substitutable residue on the cyclic peptide comprises an additional moiety attached via a side chain of the substitutable residue, or a residue substituting for the substitutable residue.

9. The MOF of claim 8, wherein one or more substitutable residue is substituted with a lysine residue, optionally
wherein one or more lysine residue is conjugated to an additional moiety.

10. The MOF of claim 7, comprising a multimer comprising (i) at least two copies of cyclic peptide EhPEhPEhP (SEQ ID NO:1) or salt thereof, wherein E can be substituted with any L amino acid that is not proline, and (ii) Co²⁺.

11. The MOF of claim 10, comprising a crystal structure of the MOF, optionally wherein in the crystal structure each Co²⁺ cation is octahedrally coordinated to three water molecules and three histidines from different peptides in a planar fashion, and the glutamates do not participate in coordination but fill the crystal pores.

12. The MOF of claim 7, comprising a multimer comprising (i) at least two copies of cyclic peptide DhmDhmDhm (SEQ ID NO:2) or salt thereof, wherein m can be any alpha D amino acid that is not proline, and (ii) Zn²⁺.

13. The MOF of claim 12, wherein the MOF comprises a crystal structure of the MOF, optionally wherein in the crystal structure each Zn²⁺ cation is internally coordinated with three histidines from one peptide and an aspartic acid from an adjacent peptide.

14. The MOF of claim 7, comprising a multimer comprising (i) at least two copies of cyclic peptide (3-(4-Pyridyl)-alanine-β-Homoproline-α-Aminobutyric acid-3-(4-Pyridyl)-alanine-β-Homoproline-α-Aminobutyric acid), or salt thereof, wherein D-α-Aminobutyric acid can be substituted with any D amino acid that is not proline (SEQ ID NO:3), and (ii) Zn²⁺.

15. The MOF of claim 14, wherein the MOF comprises a crystal structure of the MOF, optionally wherein in the crystal structure each Zn²⁺ cation is linked to two peptides through pyridine coordination while two water molecules fill the other positions for full tetrahedral coordination.

16. The MOF of claim 7, comprising a multimer comprising (i) at least two copies of cyclic peptide 3-(4-Pyridyl)-alanine-1,2,3,4-tetrahydroisoquinoline-3-carboxylic acid-3-Aminobutanoic acid-3-(4-Pyridyl)-alanine-1,2,3,4-tetrahydroisoquinoline-3-carboxylic acid-3-Aminobutanoic acid), or salt thereof; wherein 3-aminobutanoic acid can be substituted to any R amino acid such as β-phenylalanine, β-alanine, or 3-Aminoisobuteric acid, and (ii) Zn²⁺.

17. The MOF of claim 16, wherein the MOF comprises a crystal structure of the MOF, optionally wherein in the crystal structure each Zn²⁺ cation is tetrahedrally coordinated with two pyridine ligands and two water molecules.

18. The MOF of claim 7, comprising a multimer comprising (i) at least two copies of cyclic peptide aNkhPeAnKHpE (SEQ ID NO:5), or salt thereof; wherein a, k, h, e, and n can be substituted to any D amino acid that is not proline; and wherein N, A, K H, and E can be substituted to any L amino acid that is not proline; and (ii) Zn²⁺.

19. The MOF of claim 18, wherein the MOF comprises a crystal structure of the MOF, optionally wherein in the crystal structure each Zn²⁺ cation occupies empty open channels in the crystal and are not coordinated to any of the metal-binding residues.

20. The MOF of claim 7, comprising a multimer comprising (i) at least two copies of cyclic peptide ppKvEPPkVe (SEQ ID NO:6), or salt thereof, wherein v can be substituted to any D amino acid that is not proline, and V can be substituted to any L amino acid that is not proline; and (ii) Cu²⁺; optionally
wherein the MOF comprises a crystal structure of the MOF, optionally wherein in the crystal structure each Cu²⁺ cation is coordinated between two peptides via two lysines and two glutamates in a square planar geometry.
